(19) United States
(12) Patent Application Publication
(10) Pub. No.: US 2007/0202206 A1
(43) Pub. Date: Aug. 30, 2007

(54) Morinda Citrifolia Leaf Juice And Leaf Extract Based Formulations

(76) Inventors: Afa Kehaati Palu, American Fork, UT (US); Bing-Nan Zhou, Sandy, UT (US); Chen Su, West Jordan, UT (US); Claude Jarake Jensen, Cedar Hills, UT (US); Brett West, Orem, UT (US); Stephen Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/560,407

(22) Filed: Nov. 16, 2006

Related U.S. Application Data
(60) Provisional application No. 60/740,612, filed on Nov. 29, 2005.

(51) Int. Cl. A61K 36/746

(52) U.S. Cl. 424/774; 424/776

(57) ABSTRACT

A method and composition for inhibiting certain enzymes, where such inhibition results in various health benefits is provided. More particularly, a method and composition using one or more of the following: Noni Leaf Extract; Noni Leaf Juice; and/or Roast Leaf to inhibit the following: HMG-CoA Reductase; Phosphodiesterases (3 and 4) PDE3 and PDE4; 5-Lipoxygenase (LOX) and 15-LOX; Xanthine Oxidase (XO); Gamma Amino Butyric Acid (GABA) and the growth of the second most common human skin cancer cell line, is provided. Moreover, the foregoing enzymatic inhibitions result in: alleviating pain and inflammation; treating prostate cancers; lowering cholesterol levels; counteracting Diabetes Type II; maintaining the highest possible integrity of cellular interactions in the brain resulting in an undisturbed neural function, (i.e., neuroprotection); ameliorating the effects of asthma and allergies; improving energy; improving insulin secretion; decreasing kidney stone accumulation; alleviating the effects of gout; minimizing convulsions related to epilepsy and other seizure disorders; and providing palliative effects to those addicted to drugs.

### 1. RELATED APPLICATIONS

This application claims priority to United States Provisional Application Ser. No. 60/740,612 filed Nov. 29, 2005, entitled “Morinda Citrifolia Leaf Juice and Leaf Extract Based Formulations”.

### BACKGROUND

2. Field of Invention

This invention relates to a method and composition for inhibiting certain enzymes, where such inhibition results in various health benefits. More particularly, this invention relates to using one or more of the following: Noni Leaf Extract; Noni Leaf Juice; and/or Roast Leaf to inhibit the following: HMG-CoA Reductase; Phosphodiesterases (3 and 4) PDE3 and PDE4; 5-Lipoxygenase (LOX) and 15-LOX; Xanthine Oxidase (XO); Gamma Amino Butyric Acid (GABA) and the growth of the second most common human skin cancer cell line.

Moreover, the foregoing enzymatic inhibitions result in: alleviating pain and inflammation; treating prostate cancers; lowering cholesterol levels; counteracting Diabetes Type II; maintaining the highest possible integrity of cellular interactions in the brain resulting in an undisturbed neural function, (i.e., neuroprotection); ameliorating the effects of asthma and allergies; improving energy; improving insulin secretion; decreasing kidney stone accumulation; alleviating the effects of gout; minimizing convulsions related to epilepsy and other seizure disorders; and providing palliative effects to those addicted to drugs.

3. Background

People are becoming increasingly more conscientious of their health. With a variety of deadly diseases and ailments threatening the public health each year, efforts to find treatments and medications that treat and prevent disease are ongoing. Moreover, studies show that comprehensive, novel early prevention and detection strategies increase healthy life potential such that: cancer is more preventable and curable; obesity and its consequences, such as diabetes and heart and vascular diseases, are greatly reduced; and causes of mental, neurological and behavioral diseases are better understood and managed.

Such early prevention and detection strategies include using substances that have the effect of inhibiting the growth of certain enzymes, wherein such inhibition results in improved health. In fact, the Department of Health and Human Services reports that new drugs and innovation are being rapidly approved, and such scientific advances are measurably reducing the burden of all chronic diseases.

5-LOX and 15-LOX

Two enzymes in particular, when inhibited, decrease the incidence and potential for certain diseases. Specifically, the enzymes 5- and 15-lipoxygenase (aka 5- and 15-LOX) alleviate ailments associated with pain and inflammation as well as reduce incidence of and treat certain prostate cancers.

First, understanding the mechanisms behind LOX inhibition leads to an understanding of pain and inflammation alleviation in general. And, an understanding of how to suppress pain and inflammation in areas of the body leads to an understanding of how to treat and prevent various ailments related to pain and inflammation, and especially those diseases related to prostate cancers and neural disorders (i.e., neuroprotection). Thus, the following discussion is provided to foster such understanding.

As background, 5- and 15-LOX produce active metabolites from arachidonic acid that cause inflammation. More specificially, eicosanoids are continuously synthesized in membranes 20-carbon fatty acid chains that contain at least three double bonds. There are four major classes of eicosanoids—prostaglandins, prostacyclins, thromboxanes, and leukotrienes—and they are all made mainly from arachidonic acid. The synthesis of all but the leukotrienes involves the enzyme cyclooxygenase (COX); the synthesis of leukotrienes involves the enzyme lipoxygenase (LOX). These synthetic pathways are targets for a large number of therapeutic drugs because eicosanoids play an important part in, among other things discussed below, pain, fever, and inflammation.

As examples of drugs currently available in the industry to treat pain and inflammation-related ailments, corticosteroid hormones such as cortisone, for example, inhibit the activity of the phospholipase in the first step of the eicosanoid synthesis pathway, and are widely used clinically to treat noninfectious inflammatory diseases, such as some forms of arthritis. Moreover, nonsteroid anti-inflammatory drugs such as aspirin and ibuprofen, in contrast, block the first oxidation step, which is catalyzed by cyclooxygenase. Certain prostaglandins that are produced in large amounts in the uterus at the time of childbirth to stimulate the contraction of the uterine smooth muscle cells are widely used as pharmacological agents to induce abortion. Though many drugs currently available reduce pain and inflammation, they also have undesirable side effects.

In further detail, the enzymes of the 5-LOX and 15-LOX pathway produce active metabolites from arachidonic acid that cause inflammation. This has been shown both by the identification of higher levels of leukotrienes in both acute and chronic inflammatory lesions coupled with the evidence of primary signs of inflammation when leukotrienes are added to tissue cultures. Leukotrienes are a family of lipid mediators involved in acute and chronic inflammation and allergic response diseases. They are the biologically active metabolites of arachidonic acid and have been implicated in the pathological manifestations of inflammatory diseases, including asthma, arthritis, psoriasis, and inflammatory bowel disease. The biosynthesis of leukotrienes (LT or LT's) begins with the oxygenation of arachidonic acid into an unstable epoxide known as LTA4 (an intermediate central to the formation of leukotrienes) by the enzyme 5-lipoxygenase (5-LOX). LTA4 can further be converted into the potent chemo attractant LTB4 by the enzyme LTA4 hydrolase or conjugated with glutathione (GSH) to produce LTC4 by a specific microsomal GSH S-transferase (MGST) known as LTC4 synthetase (LTC4S). LTC4 is the parent compound of the cysteinyl-leukotrienes (cys-LTs) that include LTC4, LTD4, and LTE4. These three cysteinyl-leukotrienes are potent smooth muscle constricting agents, particularly in the respiratory and circulatory systems. These are mediated via at least two cell receptors, CysLT1 and CysLT2. The CysLT1 receptor is a G-protein-coupled receptor with seven transmembrane regions. A large amount of data that has been collected, which clearly demonstrates that the CysLT's play a pivotal role in inflammatory and allergic response diseases, particularly asthma.

It has also been established that these lipid mediators have profound hemodynamic effects, constricting coronary blood vessels, resulting in a reduction of cardiac output efficiency. Moreover, CysLT's have been shown to induce the secretion of von Willebrand factor and surface expression of P-selectin in cultured HUVEC. Von Willebrand is a genetic disorder. The most common types, and those most familiar to people, are the hemophiliac diseases.

In addition, the cysteinyl LT's are predominantly secreted by eosinophils, mast cells, and macrophages, which cause vasodilatation, increase postcapillary venule permeability, and stimulate bronchoconstriction and mucous secretion. Furthermore, it has been observed that elevated leukotriene LTC4 synthase activity was observed in peripheral blood granulocyte suspensions from patients with chronic myeloid leukemia (CML), and human bone marrow-derived myeloid progenitor cells. In asthma, the cysteinyl leukotrienes are present in alveolar lavage fluid of patients.

Hence, the presence of 5-LOX and leukotriene synthase are clinically important in the diagnosis of patients with bronchial asthma as well as to patients suffering from those ailments previously discussed. As a result, 5-LOX inhibition would offer these patients alleviation or treatment from their ailments or diseases.

Additionally, eicosanoids, (which again, are generated by the metabolism of arachidonic acid via the LOX pathway), have also been implicated in the pathogenesis of prostate cancer. LOX expression fosters tumor promotion, progression, and metastasis. And, involvement of LOXs expression and function in tumor growth and metastasis has been reported in human tumor cell lines. Expressions of 5-LOX in prostate cancer patients, and normal prostate tissues were examined, as well as effects of their inhibitors on cell proliferation in two prostate cancer cell lines (PC3, DU-145).¹ Expression of 5-LOX protein was detected by immunohistochemistry. Effects of LOX inhibitors on prostate cancer cell growth caused marked reduction of prostate cancer cells in a concentration- and time-dependent manner.² In fact, the LOX inhibitors caused marked inhibition of PC cells through apoptosis. Hence, because LOX is induced in prostate cancer, LOX inhibitors may mediate potent antiproliferative effects against prostate cancer cells. In other words, LOX is a relatively new target in treatment of prostate cancer and 5-LOX inhibition may provide a viable treatment.
¹1Int J Oncol. 2004 Apr;24(4):821-7. “Expression of lipoxygenase in human prostate cancer and growth reduction by its inhibitors.” Matsuyama M, Yoshimura R, Mitsuhashi M, Hase T, Tsuchida K, Takemoto Y, Kawahito Y, Sano H, Nakatani T. Department of Urology, Osaka City University Graduate School of Medicine, Osaka 545-8585, Japan.

²Id.

Furthermore, 5-LOX is expressed in the brain, including in CNS neurons. The physiologic role may be inflammation associated with tumor growth in the brain. In fact, high 5-LOX expression was detected in the proliferating brain cells and thus, reduction in tumor cell proliferation and/or destruction of tumor cells is achievable with 5-LOX inhibitors.³
³1: Ann N Y Acad Sci. 2001 Jun;939:45-51. “Neurogenesis and neuroprotection in the adult brain. A putative role for 5-lipoxygenase?”Manev H, Uz T, Manev R, Zhang Z.

As a result, it is advantageous to provide substances and compositions that inhibit the 5- and 15- LOX enzymes and consequently, measurably reduce the burden of the foregoing diseases and ailments. Moreover, it is additionally advantageous to provide substances and compositions that inhibit 5- and 15-LOX and make possible the prevention and treatment diseases, such as: various cancers; nervous system disorders, and a medley of other diseases.

HMG-CoA Reductase

To understand how HMG-CoA inhibition lowers cholesterol, the following background is provided. Cholesterol is a fatty lipid found in the body tissues and blood plasma of vertebrates. Cholesterol can be found in large concentrations in the brain, spinal cord, and liver. The liver is the most important site of cholesterol biosynthesis, although other sites include the adrenal glands and reproductive organs. The insolubility of cholesterol in water is a factor in the development of atherosclerosis, the pathological deposition of plaques of cholesterol and other lipids on the insides of major blood vessels, a condition associated with coronary artery disease.

Recent research shows that the relative abundance lipoproteins, to which cholesterol becomes attached may be the real cause of cholesterol buildup in the blood vessels. High-density lipoprotein (HDL) carries cholesterol out of the bloodstream for excretion, while low-density lipoprotein (LDL) carries it back into the system for use by various body cells. Researchers believe that HDL and LDL levels in the bloodstream may be at least as important as cholesterol levels, and now measure both to determine risk for heart disease.

Maintaining a healthy cholesterol level in the blood is crucial to the health of many living organisms, including human beings. Cholesterol synthesis can be effectively blocked by a class of compounds called statins. Statins are potent competitive inhibitors (Ki<1 nM) of HMG-CoA reductase, the essential control point in the biosynthetic pathway. HMG-CoA Reductase is particularly responsible for cholesterol synthesis. Inhibition of HMG-CoA Reductase decreases excess cholesterol production. Plasma cholesterol levels decrease by 50% in many patients given both statin and inhibitors of bile-salt reabsorption. Inhibitors of HMG-CoA reductase are widely used to lower the plasma cholesterol level in people who have atherosclerosis, which is the leading cause of death in industrialized societies.

A study reported in 1998 that HMG-CoA Reductase inhibitors protect against stroke through endothelial nitric oxide synthase. Treatment of ischemic strokes is limited to prophylactic agents that block the coagulation cascade so that no plaque forms inside the arteries. Plaque formations inside arteries reduce arterial volume and restrict blood flow, thereby increasing the blood pressure to abnormal levels. This study showed for the first time that HMG-CoA Reductase inhibitors are cholesterol-lowering agents that protect against cerebral injury by an unidentified mechanism that involves the selective up-regulation of endothelial NO synthase (eNOS). The prophylactic treatment with HMG-CoA Reductase inhibitors augments cerebral blood flow, thus reducing cerebral infarct size and ultimately improves the neurological functions in normocholesterolemic mice. This study concluded that HMG-CoA Reductase inhibitors provide a prophylactic treatment strategy for increasing blood flow and reducing brain injury during cerebral ischemia (localized tissue anemia due to obstruction of the inflow of arterial blood).

Another study reported in 2003 the effects of HMG-CoA Reductase inhibitors on cardiovascular diseases. This study found that HMG-CoA Reductase inhibitors lower the level of circulating LDL cholesterol by blocking the action of HMG-CoA Reductase. In several clinical trials, the following additional benefits were discovered in addition to the cholesterol lowering benefits: improvements in vasoreactivity, homeostasis, plaque stability, reduction of proinflammatory events such as decreases in monocyte adhesion and infiltration. These benefits account for why statins help to treat or prevent cardiovascular diseases.

Many types of statins on the market are designed to inhibit the HMG-CoA Reductase enzyme. Some of the drugs are synthetic, and some are derived from natural sources, such as from fungi. Some examples of the statins or HMG-CoA Reductase inhibitors include: Lovastatin, marketed under the brand name MEVACOR™, Simvastativ, marketed under brand name ZOCOR™, Pravastatin, marketed under the brand name PRAVACHOL™, Fluvastatin, marketed under the brand name LESCOL™, and Atorvastatin, marketed under the brand name LIPITOR™.

As noted above, statins have many beneficial effects for the human body. However, as with many drugs, statins also have various known undesirable side effects. For example, some common side effects of existing statins include: muscle tenderness or soreness, unexplained muscle pain, general malaise, fatigue and weakness, fever, weakness, and flu-like illness. Moreover, statins generally are not recommended for those who have liver diseases, are pregnant or planning to be pregnant, are breast feeding, or who drink more than 1-2 alcoholic drinks per day.

In 2002, it was reported that homocysteine induces the unregulated expression of the HMG-CoA Reductase enzyme, which increases the production of cholesterol in the body. Consequently, homocysteine suppresses the production of nitric oxide. The administration of statin and statin-like drugs reduces cholesterol synthesis and improves endothelial function, thereby restoring cardiovascular health.

Elevated blood cholesterol is one of the major modifiable risk factors for coronary heart disease (CHD), the leading cause of death in the U.S. CHD accounts for approximately 490,000 deaths each year, and angina and nonfatal myocardial infarction (MI) are a source of substantial morbidity. CHD is projected to cost over $60 billion in 1995 in the U.S. in medical expenses and lost productivity. Clinical events are the result of a multifactorial process that begins years before the onset of symptoms. Autopsy studies detected early lesions of atherosclerosis in many adolescents and young adults. The onset of atherosclerosis and symptomatic CHD is earlier among persons with inherited lipid disorders such as familial hypercholesterolemia (FH) and familial combined hyperlipidemia (FCH).

Epidemiologic, patho-logic, animal, genetic, and clinical studies support a causal relationship between blood lipids (usually measured as serum levels) and coronary atherosclerosis. High cholesterol is a risk factor for CHD. Because CHD is a multifactorial process, however, there is no definition of high cholesterol that discriminates well between individuals who will or will not develop CHD. The risk associated with high total cholesterol is primarily due to high levels of low-density lipoprotein cholesterol (LDL-C), but there is a strong, independent, and inverse association between high-density lipoprotein cholesterol (HDL-C) levels and CHD risk.

Thus, it is advantageous to provide a substance that inhibits the HMG-CoA Reductase enzyme, and thereby, inhibits excess cholesterol without the risk of many unhealthy side effects.

PDE3 and PDE4

Phosphodiesterases, also known as PDEs, are a group of enzymes found in the human body. PDEs react with a chemical, cAMP, in the body and cAMP is needed for normal cell function.

Among the many reasons it is beneficial to inhibit PDEs include: to alleviate the suffering of asthma and allergy patients, and to boost body energy. Specifically, an allergy is an excessive response to allergens in one or more parts of the body. Similarly, asthma, an airway disease, is due to the excessive response in the airway. An allergy begins when antigens enter the body tissues, these cells meet antigens, and IgE antibodies are released. The IgE antibodies bind to mask cells in tissues. This, combined with the secretion of histamine results in an allergy. Thus controlling the release of IgE will stop an allergy.

Research shows that cAMP controls the release of IgE. Using an inhibiting PDE increased the level of cAMP in the body. Thus a PDE inhibitor will help allergy and asthma patients.

PDE inhibitors also help asthma patients during an attack. During an asthma attack, the muscles around the airway are constricted and inflamed, and cAMP relaxes the muscles around the airways. Thus, inhibiting PDEs increases the level of cAMP, helping to relax the muscles around the airways and helping to fight and treat an asthma attack.

A PDE inhibitor also boosts body energy. The body produces energy by using glucose. A body in need of energy produces glucose by converting glucagons. This conversion process includes both liver cells and fat cells that include cAMP. The PDE inhibitor boosts body energy by increasing the level of cAMP.

Similarly, PDE inhibitors can increase insulin secretion and thus alleviate and treat symptoms associated with Type II Diabetes. The American Diabetes Association estimates that 91 percent of the persons who have diabetes have Type II diabetes. Type I diabetes, an autoimmune disease, results from pancreatic β cell destruction in which insulin secretion is virtually absent, and Type II diabetes, in contrast, is due to impaired insulin secretion of varying severity and inability to utilize insulin properly (insulin resistance). Insulin granules are secreted from pancreatic β cells. There are two kinds of ion channels involved in controlling insulin release, potassium and calcium channels. When the blood glucose level is elevated, the potassium channels close and calcium channels open, leading to an influx of calcium ions into the β cells. The resulting increase in calcium ions in β cells triggers the release of insulin. When the blood glucose level drops, the potassium channels reopen and the release of insulin decreases. Since PDE inhibitors increase insulin secretion, they also serve to treat and alleviate Type II Diabetes-related ailments.

Thus one skilled the art will appreciate that PDE inhibitors are beneficial for the reasons explained above.

Xanthine Oxidase

Many diseases or symptoms of diseases arise from a deficiency or excess of a specific metabolite in the body. Xanthine oxidase (XO) is a flavoprotein enzyme, found in most species (from bacteria to human). In general XO catalyses the oxidative hydroxylation of purine substrates and subsequent reduction of O2 at the flavin centre with generation of reactive oxygen species (ROS), either superxoide anion radical or hydrogen peroxide (the oxidative half-reaction). One of the specific reactions in which XO participates is the oxidation of hypoxanthine and the oxidation of xanthine. XO catalyses the oxidation of hypoxanthine to xanthine and then to uric acid. Uric acid plays an important role in gout. Inhibition of XO decreases the uric acid levels, and results in an antihyperuricemic effect. Allopurinol is prescribed as a xanthine oxidase inhibitor used in the treatment of gout and gout-like diseases. XO serum levels are significantly increased in various pathological states like hepatitis, inflammation, ischemia reperfusion, carcinogenesis and aging and the ROS generated in the enzymatic process are involved in oxidative damage. Moreover, XO inhibition may also decrease kidney stone accumulation and improve a mammal's cardiovascular health.

It is therefore possible that the inhibition of this enzymatic pathway would be therapeutically beneficial.

GABA⁴
⁴ Information taken from article by Leah Ariniello, Science Writer, Society for Neuroscience, 11 Dupont Circle, NW, Suite 500, Washington D.C., 20036. August 1996.© Society of Neuroscience

GABA is a chemical that stands for gamma-amino butyric acid. Abnormal levels of GABA cause seizures, so many health professionals seek substances that inhibiting this enzyme to lower the incidence of seizures and convulsion activity. That is, new treatments that promote harmony in the message delivery system and efficiently ward off seizures are desirable.

Specifically, epilepsy is the disease of people who experience repeated seizures. Experts suggest this ailment is one of the most prevalent neurological disorders, afflicting approximately one percent of Americans—about 2.5 million people. Seizures can occur in healthy children, adults and the elderly as well as those with other disorders such as a brain injury.

Traditional medications are not successful for all patients and carry numerous side effects. Thus, it is desirable to provide substances that inhibit GABA with little or no side effects.

GABA also has central nervous system implications. In other words, GABA is a CNS depressant. And, when released into the synapses, GABA binds to receptors and slows down brain activity. So, when GABA is inhibited, it offers palliative effects to those addicted to drugs.

It is therefore advantageous to provide substances and compounds that inhibit GABA.

Inhibition of Growth of Second Most Prevalent Human Skin Cancer Cell Line⁵
⁵ Paragraph below based on article: © 1998-2005 by Michael W. Davidson, Mortimer Abramowitz, Olympus America Inc., and The Florida State University. All Rights Reserved.

Human epidermoid carcinoma A431 cells are the second most prevalent skin cancer cells. The A-431 cell line, is tumorigenic, and forms rapidly growing subcutaneous tumors in immunosuppressed mice and colonies in soft agar. Also known as cutaneous squamous cell carcinoma, A431 (i.e., epidermoid carcinoma of the skin) is a malignant tumor of epidermal keratinocytes, a condition that has increased in frequency considerably over the last century. Some say this is due in part to the ongoing depletion of the protective ozone layer.

It is therefore advantageous to provide substances or compositions that inhibit the growth of this carcinoma.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates the percent growth of tumor cells in the presence of leaf extract.

FIG. 2 illustrates percent growth of tumor cells in the presence of leaf extract; and

FIG. 3 illustrates illustrates tumor skin cell growth in the presence of Miotomycin.

### SUMMARY AND OBJECTS OF THE INVENTION

Some embodiments of the present invention provide a method for and composition for inhibiting the HMG-CoA Reductase enzyme; PDE3 and PDE4 enzymes; 5- and 15-LOX enzymes; XO enzyme; GABA enzyme and for inhibiting the growth of the second most found human skin cancer cell line.

Some embodiments may result in: alleviating pain and inflammation; treating prostate cancers; lowered cholesterol levels; counteracting Diabetes Type II; maintaining the highest possible integrity of cellular interactions in the brain resulting in an undisturbed neural function, (i.e., neuroprotection); ameliorating the effects of asthma and allergies; improving energy and insulin secretion; decreasing kidney stone accumulation; alleviating the effects of gout; minimizing convulsions related to epilepsy and other seizure disorders; and providing palliative effects to those addicted to drugs.

Some embodiments of the present invention provide a method of treating various diseases and ailments, which comprise administering to said mammal a compound containing an effective amount of either Noni leaf juice, Noni Leaf Extract or Roast Leaf, wherein said compound inhibits 5-Lox and 15-Lox, HMG-CoA Reductase enzyme; PDE3 and PDE4 enzymes; 5- and 15-LOX; XO; and GABA.

5- and 15-LOX

In a preferred embodiment, the present invention comprise various methods of using specially processed components of the Indian Mulberry or Morinda citrifolia L. plant to inhibit the oxygenation and metabolizing of arachidonic acid into its leukotriene synthesized intermediates by inhibiting 5-Lipoxygenase (5-LOX), 15-Lipoxygenase (15-LOX) and the lipid mediators known as leukotrienes that contribute to the pathological manifestations of inflammatory diseases, to the treatment and prevention of prostate cancers, and to maintaining the highest possible integrity of cellular interactions in the brain resulting in an undisturbed neural function, (i.e., neuroprotection), as well as the treatment and prevention of other diseases.

In the preferred embodiment, the present invention comprises various methods utilizing a composition including one or more processed Morinda citrifolia components such as: leaf extracts, leaf juice, and defatted and untreated seed extracts. Some of these methods include the steps of administering a Morinda citrifolia composition to a mammal to inhibit, prevent, or treat inflammatory diseases, neuro-diseases, or prostate cancers.

In some embodiments the present composition of the present invention comprises Morinda citrifolia leaf juice or leaf extract, formulated with or without other ingredients, either natural or artificial, as needed.

HMG-CoA Reductase

In a preferred embodiment the invention comprises methods and compositions for improving profiles of lipoproteins, reducing VLDL and LDL lipoprotein levels, increasing HDL lipoprotein levels, decreasing the absorption of fatty acids across the intestinal epithelium, inhibiting HMG-CoA Reductase and reducing total blood cholesterol in living organisms utilizing Noni Leaf Juice, Noni Leaf Extract or other Noni Leaf products derived from Morinda citrifolia L. The invention includes methods and compositions for selectively decreasing LDLs.

Embodiments of the present invention comprise methods and compositions for inhibiting HMG-CoA Reductase without causing the negative side effects associated with statins currently available on the market.

Specifically, the formulations of the invention comprise processed Morinda citrifolia products. In one embodiment, the formulations include a composition including one or more processed Morinda citrifolia components such as: leaf extracts, leaf juice, and defatted and untreated seed extracts. Some of these methods include the steps of administering a Morinda citrifolia composition to a mammal to inhibit HMG-CoA Reductase and thereby, lower cholesterol.

In some embodiments the present composition of the present invention comprises Morinda citrifolia leaf juice or leaf extract, formulated with or without other ingredients, either natural or artificial, as needed.

Some embodiments of the invention provide methods of inhibiting the activity of HMG-CoA Reductase without causing the negative secondary effects caused by known statins.

Some embodiments of the invention provide an orally administered HMG-CoA Reductase inhibitor capable of use during pregnancy.

Some embodiments of the invention provide an orally administered composition capable of inhibiting HMG-CoA Reductase activity in patients that do not respond to known statins.

Some embodiments of the invention provide an over-the-counter composition for inhibiting HMG-CoA Reductase activity in mammals without requiring a prescription.

PDE3 and PDE4

In a preferred embodiment, the present invention relates to methods and formulations for using Noni Leaf Extract and Noni Leaf Juice for inhibiting naturally occurring phosphodiesterases (PDEs). In other words, the present invention relates to Noni leaf extract and Noni leaf juice as a PDE inhibitor.

The present invention comprises Noni leaf juice and Noni leaf extract compositions, each of which include one or more extracts from the Morinda citrifolia L. plant in an amount capable of maximizing the inhibition of the PDE enzyme without causing negative side effects when the composition is administered to a mammal.

In some embodiments the present composition of the present invention comprises Morinda citrifolia leaf juice or leaf extract, formulated with or without other ingredients, either natural or artificial, as needed.

XO

In a preferred embodiment, a method and composition for utilizing Noni Leaf Juice and Noni Leaf extracts, is provided, which inhibits the enzyme, xanthine oxidase to ameliorate gout and gout-like diseases, to decrease kidney stone accumulation and to improve a mammal's overall health.

Noni leaf juice and Noni leaf extracts when provided to a mammal in sufficient amounts act as a useful anthine oxidase inhibitor. The inhibition of the enzyme function results in a decrease in the uric acid levels resulting in an antihyperuricemic effect. This is useful in treating diseases caused by an excess of uric acid such as gout.

Some of these methods include the steps of administering a Morinda citrifolia composition to a mammal to inhibit, prevent, or treat gout-like diseases, to decrease kidney stone accumulation and improve cardiovascular health.

In some embodiments the present composition of the present invention comprises Morinda citrifolia leaf juice or leaf extract, formulated with or without other ingredients, either natural or artificial, as needed.

GABA

In a preferred embodiment the invention comprises methods and compositions for normalizing levels of GABA to prevent seizures and to treat and alleviate the effects of epilepsy by inhibiting the amount of GABA that is expressed in a mammal. Specifically, in the preferred embodiment, a composition utilizing Noni Leaf Juice, Noni Leaf Extract or other Noni Leaf products derived from Morinda citrifolia L is provided that inhibits GABA expression to normalize levels of GABA, which thereby reduces convulsant activity and treats seizures.

Embodiments of the present invention comprise methods and compositions for inhibiting GABA without causing the negative side effects associated with medication currently available for seizure-sufferers or mammals with epileptic-type diseases.

Specifically, the formulations of the invention comprise processed Morinda citrifolia products. In one embodiment, the formulations include a composition including one or more processed Morinda citrifolia components such as: leaf extracts, leaf juice, and defatted and untreated seed extracts. Some of these methods include the steps of administering a Morinda citrifolia composition to a mammal to inhibit GABA and thereby, lower the incidence and effects of epilepsy and seizures.

In some embodiments the present composition of the present invention comprises Morinda citrifolia leaf juice or leaf extract, formulated with or without other ingredients, either natural or artificial, as needed.

Some embodiments of the invention provide methods of inhibiting the activity of GABA without causing the negative secondary effects.

Some embodiments of the invention provide an orally administered GABA inhibitor capable of use during pregnancy.

Some embodiments of the invention provide an orally administered composition capable of inhibiting GABA activity in patients that do not respond to other known GABA inhibitors.

Some embodiments of the invention provide an over-the-counter composition for inhibiting GABA activity in mammals without requiring a prescription.

Some embodiments of the invention provide palliative treatment to those suffering from drug addiction through use of GABA inhibitors.

Skin Cancer Growth Inhibition

In a preferred embodiment of the present invention, Noni leaf juice and Noni leaf extract cause significant inhibitions in the growth of the second most prevalent human skin cancer cell lines.

In the preferred embodiment, the present invention comprises various methods utilizing a composition including one or more processed Morinda citrifolia components such as: leaf extracts, leaf juice, roast leaf, and defatted and untreated seed extracts. Some of these methods include the steps of administering a Morinda citrifolia composition to a mammal to inhibit the growth of the A431 human skin cancer cell line.

In some embodiments the present composition of the present invention comprises Morinda citrifolia leaf juice or leaf extract, formulated with or without other ingredients, either natural or artificial, as needed.

Moreover, a preferred embodiment of the present invention provides the presence of isoflavones found in dried Noni leaf. Specifically, Daidzin was found at 2.51 mg per 100 grams; Gycitin was found at 1.85 mg per 100 g; and Genistin was found at 3.13 mg per 100 grams.⁶ Additionally, the presence of sterols in Tahitian Noni Fruit Juice concentrate was also found: campesterol was found at 12 mg per 100 grams; stigmasterol was found at 8.4 mg per 100 grams; Beta Sitosterol was found at 21.5 mg per 100 grams.
⁶ Klump et al. Journal of AOAC International Vol 84, No. 6. 2001.

Isoflavones appear to protect against hormone-related disorders such as breast cancer and prostate cancers. Research in several areas of healthcare has shown that consumption of isoflavones may play a role in lowering risk for disease. Isoflavones can fight disease on several fronts.

The features and advantages may be realized and obtained by means of the instruments and combinations particularly pointed out in the appended claims. Furthermore, the features and advantages of the invention may be learned by the practice of the invention or will be obvious from the description, as set forth hereinafter.

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

It will be appreciated by those of ordinary skill in the art that the objects of this invention can be achieved without the expense of undue experimentation using well known variants, modifications, or equivalents of the methods and techniques described herein. The skilled artisan will also appreciate that alternative means, other than those specifically described, are available in the art to achieve the functional features of the molecules described herein. It is intended that the present invention include those variants, modifications, alternatives, and equivalents which are appreciated by the skilled artisan and encompassed by the spirit and scope of the present disclosure.

Embodiments of the present invention feature methods and compositions for inhibiting various enzymes, such as: HMG-CoA Reductase; PDE3 and PDE4; 5- and 15-LOX; XO; and GABA to: treat and prevent mammalian inflammatory diseases; treat prostate cancers; lower cholesterol levels; counteract Diabetes Type II; maintain the highest possible integrity of cellular interactions in the brain resulting in an undisturbed neural function, (i.e., provide neuroprotection); ameliorate the effects of asthma and allergies; improve energy and insulin secretion; decrease kidney stone accumulation; alleviate the effects of gout; minimize convulsions related to epilepsy and other seizure disorders; and provide palliative effects to those addicted to drugs. Moreover, embodiments of the present invention feature methods and compositions for inhibiting the growth of the second most found human skin cancer cell line. The foregoing list of ailments and diseases are mitigated, and the enzymatic inhibitions are fostered, through the administration of a composition comprising Noni leaf juice, Noni leaf extract and other components derived from the Indian Mulberry or Morinda citrifolia L. plant.

General Description of the Morinda citrifolia L. Plant

The Indian Mulberry or Morinda citrifolia plant, known scientifically as Morinda Citrifolia L. (“Morinda citrifolia”), is a shrub or small tree up to 10 m in height. The leaves are oppositely arranged with an elliptic to ovate form. The small white flowers are contained in a fleshy, globose, head like cluster. The fruits are large, fleshy, and ovoid. At maturity, they are creamy white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit isjuicy, bitter, dull-yellow or yellowish-white, and contains numerous red-brown, hard, oblong-triangular, winged 2-celled stones, each containing four seeds. When fully ripe, the fruit has a pronounced odor like rancid cheese. Although the fruit has been eaten by several nationalities as food, the most common use of the Morinda citrifolia plant has traditionally been as a red and yellow dye source.

The Morinda citrifolia plant is rich in natural ingredients. Those ingredients that have been discovered include from the leaves: alanine, anthraquinones, arginine, ascorbic acid, aspartic acid, calcium, beta carotene, cysteine, cystine, glycine, glutamic acid, glycosides, histidine, iron, leucine, isoleucine, methionine, niacin, phenylalanine, phosphorus, proline, resins, riboflavin, serine, beta sitosterol, thiamine, threonine, tryptophan, tyrosine, ursolic acid, and valine; from the flowers: acacetin 7 o beta d (+) glucopyranoside, 5,7 dimethyl apigenin 4′ o beta d(+) galactopyranoside, and 6,8 dimethoxy 3 methylanthraquinone 1 o beta rhamnosyl glucopyranoside; from the fruit: acetic acid, asperuloside, butanoic acid, benzoic acid, benzyl alcohol, 1 butanol, caprylic acid, decanoic acid, (E) 6 dodeceno gamma lactone, (Z,Z,Z) 8,11,14 eicosatrienoic acid, elaidic acid, ethyl decanoate, ethyl hexanoate, ethyl octanoate, ethyl palmitate, (Z) 6 (ethylthiomethyl) benzene, eugenol, glucose, heptanoic acid, 2 heptanone, hexanal, hexanamide, hexanedioic acid, hexanoic acid (hexoic acid), 1 hexanol, 3 hydroxy 2 butanone, lauric acid, limonene, linoleic acid, 2 methylbutanoic acid, 3 methyl 2 buten 1 o1, 3 methyl 3 buten 1 o1, methyl decanoate, methyl elaidate, methyl hexanoate, methyl 3 methylthio propanoate, methyl octanoate, methyl oleate, methyl palmitate, 2 methylpropanoic acid, 3 methylthiopropanoic acid, myristic acid, nonanoic acid, octanoic acid (octoic acid), oleic acid, palmitic acid, potassium, scopoletin, undecanoic acid, (Z,Z) 2,5 undecadien 1 o1, and vomifol; from the roots: anthraquinones, asperuloside (rubichloric acid), damnacanthal, glycosides, morindadiol, morindine, morindone, mucilaginous matter, nor damnacanthal, rubiadin, rubiadin monomethyl ether, resins, soranjidiol, sterols, and trihydroxymethyl anthraquinone monomethyl ether; from the root bark: alizarin, chlororubin, glycosides (pentose, hexose), morindadiol, morindanigrine, morindine, morindone, resinous matter, rubiadin monomethyl ether, and soranjidiol; from the wood: anthragallol 2,3 dimethylether; from the tissue culture: damnacanthal, lucidin, lucidin 3 primeveroside, and morindone 6beta primeveroside; from the plant: alizarin, alizarin alpha methyl ether, anthraquinones, asperuloside, hexanoic acid, morindadiol, morindone, morindogenin, octanoic acid, and ursolic acid.

Processing Morinda citrifolia Leaves

The leaves of the Morinda citrifolia plant are one possible component of the Morinda citrifolia plant that may be present in some compositions of the present invention. For example, some compositions comprise leaf extract and/or leaf juice as described further herein. Some compositions comprise a leaf serum that is comprised of both leaf extract and fruit juice obtained from the Morinda citrifolia plant. Some compositions of the present invention comprise leaf serum and/or various leaf extracts as incorporated into a nutraceutical product (“nutraceutical” herein referring to any drug or product designed to improve the health of living organisms such as human beings or mammals).

In some embodiments of the present invention, the Morinda citrifolia leaf extracts are obtained using the following process. First, relatively dry leaves from the Morinda citrifolia L. plant are collected, cut into small pieces, and placed into a crushing device—preferably a hydraulic press—where the leaf pieces are crushed. In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

In some embodiments of the present invention, the primary leaf extract is pasteurized to at least partially sterilize the extract and destroy objectionable organisms. The primary leaf extract is pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

In some embodiments of the present invention, the pasteurized primary leaf extract is placed into a centrifuge decanter where it is centrifuged to remove or separate any remaining leaf juice therein from other materials, including chlorophyll. Once the centrifuge cycle is completed, the leaf extract is in a relatively purified state. This purified leaf extract is then pasteurized again in a similar manner as discussed above to obtain a purified primary leaf extract.

Preferably, the primary leaf extract, whether pasteurized and/or purified, is further fractionated into two individual fractions: a dry hexane fraction, and an aqueous methanol fraction. This is accomplished preferably via a gas chromatograph containing silicon dioxide and CH2Cl2-MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

Processing Morinda citrifolia Fruit

Some embodiments of the present invention include a composition comprising fruit juice of the Morinda citrifolia plant. Because the Morinda citrifolia fruit is for all practical purposes inedible, the fruit must be processed in order to make it palatable for human consumption and included in the compositions of the present invention. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another product, frozen or pasteurized. In some embodiments of the present invention, the juice and pulp can be pureed into a homogenous blend to be mixed with other ingredients. Other processes include freeze drying the fruit and juice. The fruit and juice can be reconstituted during production of the final juice product. Still other processes may include air drying the fruit and juices prior to being masticated.

In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, but preferably for 2 to 3 days. The fruit is ripened or aged by being placed on equipment so that the fruit does not contact the ground. The fruit is preferably covered with a cloth or netting material during aging, but the fruit can be aged without being covered. When ready for further processing the fruit is light in color, such as a light green, light yellow, white or translucent color. The fruit is inspected for spoilage or for excessive green color and firmness. Spoiled and hard green fruit is separated from the acceptable fruit.

The ripened and aged fruit is preferably placed in plastic lined containers for further processing and transport. The containers of aged fruit can be held from 0 to 30 days, but preferably the fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.). Another product manufactured is Morinda citrifoliapuree and puree juice, in either concentrate or diluted form. Puree is essentially the pulp separated from the seeds and is different than the fruit juice product described herein.

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

Processing Morinda citrifolia Seeds

Some Morinda citrifolia compositions of the present invention include seeds from the Morinda citrifolia plant. In some embodiments of the present invention, Morinda citrifolia seeds are processed by pulverizing them into a seed powder in a laboratory mill. In some embodiments, the seed powder is left untreated. In some embodiments, the seed powder is further defatted by soaking and stirring the powder in hexane—preferably for 1 hour at room temperature (Drug:Hexane-Ratio 1:10). The residue, in some embodiments, is then filtered under vacuum, defatted again (preferably for 30 minutes under the same conditions), and filtered under vacuum again. The powder may be kept overnight in a fume hood in order to remove the residual hexane.

Still further, in some embodiments of the present invention, the defatted and/or untreated powder is extracted, preferably with ethanol 50% (m/m) for 24 hours at room temperature at a drug solvent ratio of 1:2.

Processing Morinda citrifolia Oil

Some embodiments of the present invention may comprise oil extracted from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

Compositions and Their Use

The present invention features compositions and methods for inhibiting HMG-CoA Reductase; PDE3 and PDE4; 5- and 15-LOX; XO; GABA and skin cancer. The present invention also features compositions and methods for: alleviating pain and inflammation; treating prostate cancers; lowered cholesterol levels; counteracting Diabetes Type II; maintaining the highest possible integrity of cellular interactions in the brain resulting in an undisturbed neural function, (i.e., neuroprotection); ameliorating the effects of asthma and allergies; improving energy; improving insulin secretion; decreasing kidney stone accumulation; alleviating the effects of gout; minimizing convulsions related to epilepsy and other seizure disorders; and providing palliative effects to those addicted to drugs. Embodiments of the present invention also comprise methods for internally introducing a Morinda citrifolia composition into the body of a mammal. Several embodiments of the Morinda citrifolia compositions comprise various different ingredients, each embodiment comprising one or more forms of a processed Morinda citrifolia component as taught and explained herein.

Compositions of the present invention may comprise any of a number of Morinda citrifolia components such as: leaf extract, leaf juice, leaf serum, fruit juice, fruit pulp, pulp extract, puree, seeds (whether defatted or untreated), and oil. Compositions of the present invention may also include various other ingredients. Examples of other ingredients include, but are not limited to: artificial flavoring, other natural juices or juice concentrates such as a natural grape juice concentrate or a natural blueberry juice concentrate; carrier ingredients; and others as will be further explained herein.

Any compositions having the leaf extract from the Morinda citrifolia leaves, may comprise one or more of the following: the primary leaf extract, the hexane fraction, methanol fraction, the secondary hexane and methanol fractions, the leaf serum, or the nutraceutical leaf product.

In some embodiments of the present invention, active ingredients or compounds of Morinda citrifolia components may be extracted out using various procedures and processes commonly known in the art. For instance, the active ingredients may be isolated and extracted out using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health-benefiting functions just mentioned. Quercetin and Rutin are non-limiting examples of active ingredients that may be isolated. For example, an active ingredient, including Quercetin and Rutin, may be present in amounts by weight ranging from 0.01-10 percent of the total formulation or composition. These amounts may be concentrated as well into a more potent concentration in which they are present in amounts ranging from 10 to 100 percent.

Any components and compositions of Morinda citrifolia may be further incorporated into a nutraceutical product (again, “nutraceutical” herein referring to any drug or product designed to improve the health of living organisms such as human beings or mammals). Examples of nutraceutical products may include, but are not limited to: intravenous products, topical dermal products, wound healing products, skin care products, hair care products, beauty and cosmetic products (e.g., makeup, lotions, etc.), burn healing and treatment products, first-aid products, antibacterial products, lip balms and ointments, bone healing and treatment products, meat tenderizing products, anti-inflammatory products, eye drops, deodorants, antifungal products, arthritis treatment products, muscle relaxers, toothpaste, and various nutraceutical and other products as may be further discussed herein.

The compositions of the present invention may be formulated into any of a variety of embodiments, including oral compositions, topical dermal solutions, intravenous solutions, and other products or compositions.

Oral compositions may take the form of, for example, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia components in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain the Morinda citrifolia components in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweetening agents may include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. The concentration of sweeteners may be between from 0 to 50 percent by weight of the Morinda citrifolia composition, and more preferably between about 1 and 5 percent by weight.

Typical flavoring agents can include, but are not limited to, artificial and/or natural flavoring ingredients that contribute to palatability. The concentration of flavors may range, for example, from 0 to 15 percent by weight of the Morinda citrifolia composition. Coloring agents may include food-grade artificial or natural coloring agents having a concentration ranging from 0 to 10 percent by weight of the Morinda citrifolia composition.

Typical nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B 12, C, D, E, Folic Acid, Pantothenic Acid, Biotin, etc. Examples of minerals and trace elements include, but are not limited to, calcium, chromium, copper, cobalt, boron, magnesium, iron, selenium, manganese, molybdenum, potassium, iodine, zinc, phosphorus, etc. Herbs and botanical extracts may include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The ingredients to be utilized in a topical dermal product may include any that are safe for internalizing into the body of a mammal and may exist in various forms, such as gels, lotions, creams, ointments, etc., each comprising one or more carrier agents. The ingredients or carrier agents incorporated into systemically (e.g., intravenously) administered compositions may also comprise any known in the art.

In one exemplary embodiment, a Morinda citrifolia composition of the present invention comprises one or more of a processed Morinda citrifolia component present in an amount by weight between about 0.01 and 100 percent by weight, and preferably between 0.01 and 95 percent by weight. Several embodiments of formulations are included in U.S. Pat. No. 6,214,351, issued on Apr. 10, 2001. However, these compositions are only intended to be exemplary, as one ordinarily skilled in the art will recognize other formulations or compositions comprising the processed Morinda citrifolia product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

### EXAMPLES

The following examples illustrate some of the preventative and treatment effects of some Morinda citrifolia compositions of the present invention on HMG-CoA Reductase inhibition; PDE3 and PDE4 inhibition; 5- and 15-LOX inhibition; XO inhibition; GABA inhibition and inhibition of the growth of a certain skin cancer; through the administration of a composition comprising components of the Indian Mulberry or Morinda citrifolia L. plant and specifically, through the administration of Noni leaf juice and Noni leaf extracts.

These examples are not intended to be limiting in any way, but are merely illustrative of benefits, advantages, and remedial effects of some embodiments of the Morinda citrifolia compositions of the present invention.

### Example 1

### Noni Leaf Juice

In one example, the effects of Morinda Citrifolia leaf juice on 5-LOX and 15-LOX, HMG-CoA, PDE3 and PDE4, XO and GABA were studied. The following tables summarize the results of these studies.

TABLE 1
Example 1

| | Test | No. | | Std. |
|---|---|---|---|---|
| Enzyme | Animal | Samples | % Inhibition | Deviation |
| HMG-CoA | Rat | 2 | 10% | 29 |
| Reductase | | | | |
| | | 2 | 5% | 5 |
| | | 2 | 1% | −3 |
| Lipoxygenase 15- | Rabbit | 2 | 10% | 111 |
| LOX | | | | |
| | | 2 | 5% | 102 |
| | | 2 | 1% | 87 |
| Lipoxygenase 5- | Human | 2 | 10% | 101 |
| LOX | | | | |
| | | 2 | 5% | 85 |
| | | 2 | 1% | 41 |
| Phosphodiesterase | Human | 2 | 10% | 71 |
| PDE3 | | | | |
| | | 2 | 5% | 33 |
| | | 2 | 1% | 8 |
| Phosphodiesterase | Human | 2 | 10% | 94 |
| PDE4 | | | | |
| | | 2 | 5% | 45 |
| | | 2 | 1% | 17 |
| Phosphodiesterase | Human | 2 | 10% | 41 |
| PDE5 | | | | |
| | | 2 | 5% | 0 |
| | | 2 | 1% | 6 |
| Xanthine Oxidase | Bovine | 2 | 10% | 33 |
| | | 2 | 5% | 29 |
| | | 2 | 1% | 6 |
| GABA₂, Agonist Site | Rat | 2 | 10% | 105 |
| | | 2 | 5% | 104 |
| | | 2 | 1% | 103 |

### Example 2

### Noni Leaf Extract

In another example, Morinda citrifolia leaf extract was utilized in an inhibition assay. The results are displayed in Table 2 below.

TABLE 2
Example 2

| | | No. | % NLEX in | |
|---|---|---|---|---|
| | Test Animal | Samples | Solution | % Inhibition |
| HMG-CoA | | | | |
| Reductase | | | | |
| NLEX-P | rat | 2 | 0.1% | 54 |
| | | 2 | 0.05% | 40 |
| | | | 0.025% | 7 |
| Phophodiestrerase | | | | |
| PDE3 | | | | |
| NLEX-P | hum | 2 | 0.1% | 79 |
| | | 2 | 0.05% | 69 |
| | | 2 | 0.025% | 58 |
| Phophodiestrerase | | | | |
| PDE4 | | | | |
| NLEX-P | hum | 2 | 0.1% | 82 |
| | | 2 | 0.5% | 69 |
| | | 2 | 0.025% | 54 |
| Phophodiestrerase | | | | |
| PDE5 | | | | |
| NLEX-P | hum | 2 | 0.1% | 87 |
| | | 2 | 0.05% | 84 |
| | | 2 | 0.025% | 77 |

Example 2 (above) was based on the following parameters listed in Table 3-6:

TABLE 3
HMG-CoA Reductase

| | |
|---|---|
| Source: | Wistar Rat liver |
| Substrate: | 2.5 μM[14C]HMG-CoA |
| Vehicle: | 1% DMSO |
| Pre-Incubation Time/Temp: | 15 minutes @ 37° C. |
| Incubation Buffer: | 100 mM Potassium Phosphate, pH |
| | 7.5, 20 mM G-6-P. 2.5 mM NADP |
| | 10 mM EDTA 5 mM DTT, 14 U G- |
| | 6-P-DH |
| Quantitation Method: | Quantitation of [14C] Mevalonate |
| Significance Criteria: | ≧50% of max stimulation or inhibition |

TABLE 4
Phosphodiesterase PDE3

| | |
|---|---|
| Source: | Human platelets |
| Substrate: | 1.01 μM (PH]cAMP + cAMP) |
| Vehicle: | 1% DMSO |
| Pre-Incubation Time/Temp: | 15 minutes @ 25° C. |
| Incubation Time/Temp: | 20 minutes @ 25 ° C. |
| Incubation Buffer: | 50 mM Tris-HCL, pH 7.5.5 mM |
| | MgCl₂ |
| Quantitation Method: | Quantitation of (PH) Adenosine |
| Significance Criteria: | ≧50% of max stimulation or inhibition |

TABLE 5
Phosphodiesterase PDE4

| | |
|---|---|
| Source: | Human U937 cells |
| Substrate: | 1.01 μM (PH]cAMP + cAMP) |
| Vehicle: | 1% DMSO |
| Pre-Incubation Time/Temp: | 15 minutes @ 25° C. |
| Incubation Time/Temp: | 20 minutes @ 25° C. |
| Incubation Buffer: | 50 mM Tris-HCL, pH 7.5.5 mM |
| | MgCl₂ |
| Quantitation Method: | Quantitation of (PH) Adenosine |
| Significance Criteria: | ≧50% of max stimulation or inhibition |

TABLE 6
Phosphodiesterase PDE5

| | |
|---|---|
| Source: | Human platelets |
| Substrate: | 1.01 μM (PH]cGMP + cGMP) |
| Vehicle: | 1% DMSO |
| Pre-Incubation Time/Temp: | 15 minutes @ 25° C. |
| Incubation Time/Temp: | 20 minutes @ 25° C. |
| Incubation Buffer: | 50 mM Tris-HCL, pH 7.5.5 mM |
| | MgCl₂ |
| Quantitation Method: | Quantitation of (PH) Guanosine |
| Significance Criteria: | ≧50% of max stimulation or inhibition |

### Example 3

In this next example, Morinda citrifolia leaf juice and leaf extract was shown to significantly inhibit the growth of the second most common type of human skin cancer. In this example, assays were performed to detect changes in cell proliferation based on the ability of viable cells to cause alamarBlue to change from non-fluorescent blue to a reduced, fluorescent red form. With the results obtained from the alamarBlue reaction, cell proliferation can be quantified and metabolic activity of viable cells can be examined. Test compounds including Morinda citrifolia leaf extract, leaf juice, and roast leaf were tested for their effects on the proliferation of human epidermoid carcinoma cell line-A431 at assay concentrations from 0.01 to 100 μg/ml or 0.0001% to 1% through serial 10-fold dilutions.

In summary, it was found that the leaf extract at concentrations between 10 and 100 μg/ml, as well as the leaf juice between 0.1% and 1%, caused significant growth inhibition (<50% of growth) relative to the vehicle-treated control in the tumor cell line—whereas the roast leaf failed to show a significant effect (0.01-100 μg/ml). Significant inhibitory activity was also observed for the concurrently tested standard reference agent, Mitomycin, at <10 μM. Consequently, semi-quantitative determinations of estimated LC₅₀ (50% inhibition concentration), TGI (total growth inhibition) and LC₅₀ (50% lethal concentration) by nonlinear regression analysis were calculated. Following is a description of the materials, equipment, and methods used in the assay, as well as tables summarizing the results.

Test Substances and Concentrations.

Morinda citrifolia leaf extract, leaf juice, and roast leaf were provided by Tahitian Noni International, Inc. for in vitro anti-tumor studies. The Morinda citrifolia compounds were dissolved in sterile distilled water and then diluted with sterile distilled water to obtain initial working solutions of 10000, 1000, 100, 10, and 1 μg/ml for the leaf extract and roast leaf, as well as 100, 10, 1, 0.1 and 0.01% for the leaf juice. In testing, 100-fold dilution was made in culture media to get final assay concentrations of 100, 10, 1, 0.1 and 0.01 μg/ml, and 1, 0.1, 0.01, 0.001 and 0.0001%, respectively.

Cell Line and Culture Media.

The tumor cell line, A431 (human epidermoid carcinoma), obtained from American Type Culture Collection (ATCC CRL-1555), was incubated in an air atmosphere of 5% CO₂ at 37° C. The culture medium was used with Dulbecco's Modified Eagle's medium, 90%; Fetal Bovine Serum, 10% and supplemented with 1% Antibiotic-Antimycotic.

Chemicals.

The following chemicals were used in the assay: AlamarBlue (Biosource, USA), Antibiotic-Antimycotic (GIBCO BRL, USA), Dulbecco's Modified Eagle's Medium (GIBCO BRL, USA), Fetal Bovine Serum (HyClone, USA), and Mitomycin (Kyowa, Japan).

Equipment.

The following equipment was used in the assay: CO₂ Incubator (Forma Scientific Inc., USA), Centrifuge 5810R (Eppendorf, Germany), Hemacytometer (Hausser Scientific Horsham, USA), Inverted Microscope CK-40 (Olympus, Japan), System Microscope E-400 (Nikon, Japan), Spectrafluor Plus (Tecan, Austria), and Vertical Laminar Flow (Tsao Hsin, R.O.C.).

Methods.

The anti-proliferation for the test substances was evaluated. Aliquots of 100 μl of cell suspension (about 3×10³/well) were placed in 96-well microtiter plates in an atmosphere of 5% CO₂ at 37° C. After 24 hours, 100 μl of growth medium and 2 μl of test solution, Mitomycin or vehicle (distilled water) were added respectively per well in duplicate for an additional 72-hour incubation. Two test compounds, leaf extract and roast leaf, were evaluated at concentrations of 100, 10, 1, 0.1 and 0.01 μg/ml. The other compound, leaf juice, was evaluated at concentrations of 1, 0.1, 0.01, 0.001 and 0.0001%. At the end of incubation, 20 μl of 90% alamarBlue reagent was added to each well for another 6-hour incubation before detection of cell viability by fluorescent intensity. Fluorescent intensity was measured using a Spectrafluor Plus plate reader with excitation at 530 nm and demission at 590 nm.

IC₅₀, TGI, and LC₅₀ values were then determined. IC₅₀ (50% Inhibition Concentration) is the test compound concentration where the increase from time₀ in the number or mass of treated cells was only 50% as much as the corresponding increase in the vehicle-control at the end of the experiment. TGI (Total Growth Inhibition) is the test compound concentration where the number or mass of treated cells at the end of the experiment was equal to that at time₀. LC₅₀ (50% Lethal Concentration) is the test compound concentration where the number or mass of treated cells at the end of the experiment was half that at time₀. The measured results were calculated by the following formula:
PG(%)=100×(Mean Fₜₑₛₜ−Mean Fₜᵢₘₑ₀)/(Mean F_{ctrl}−Mean Fₜᵢₘₑ₀)

If (Mean Fₜₑₛₜ−Mean Fₜᵢₘₑ₀)<0, then
PG(%)−100×(Mean Fₜₑₛₜ−Mean Fₜᵢₘₑ₀)/(Mean Fₜᵢₘₑ₀−Mean F_{blank})

Where:
PG=percent growth
Mean Fₜᵢₘₑ₀=The average of 2 measured fluorescent intensities of reduced alamarBlue at the time just before exposure of cells to the test substance.
Mean Fₜₑₛₜ=The average of 2 measured fluorescent intensities of alamarBlue after 72-hour exposure of cells to the test substance.
Mean F_{ctrl}, =The average of 2 measured fluorescent intensities of alamarBlue after 72-hour incubation without the test substance.
Mean F_{blank}=The average of 2 measured fluorescent intensities of alamarBlue in medium without cells after 72-hour incubation.

A decrease of 50% or more (≧50%) in fluorescent intensity relative to the vehicle-treated control indicated significant cell growth inhibition, cytostatic or cytotoxic activity, and a semi-quantitative IC₅₀, TGI, and LC₅₀ were then determined by nonlinear regression using GraphPad Prism (GraphPad Software, USA).

Results.

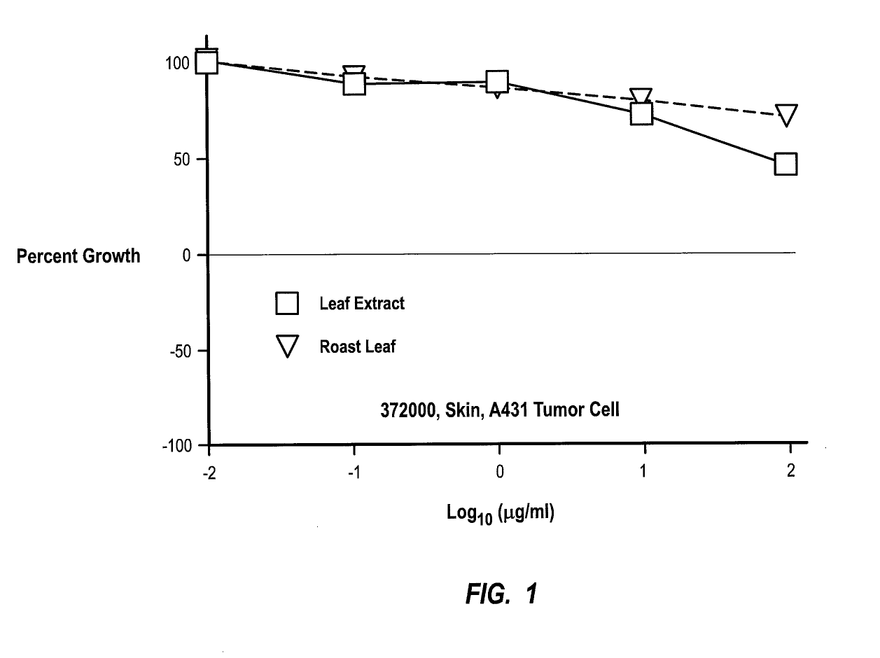

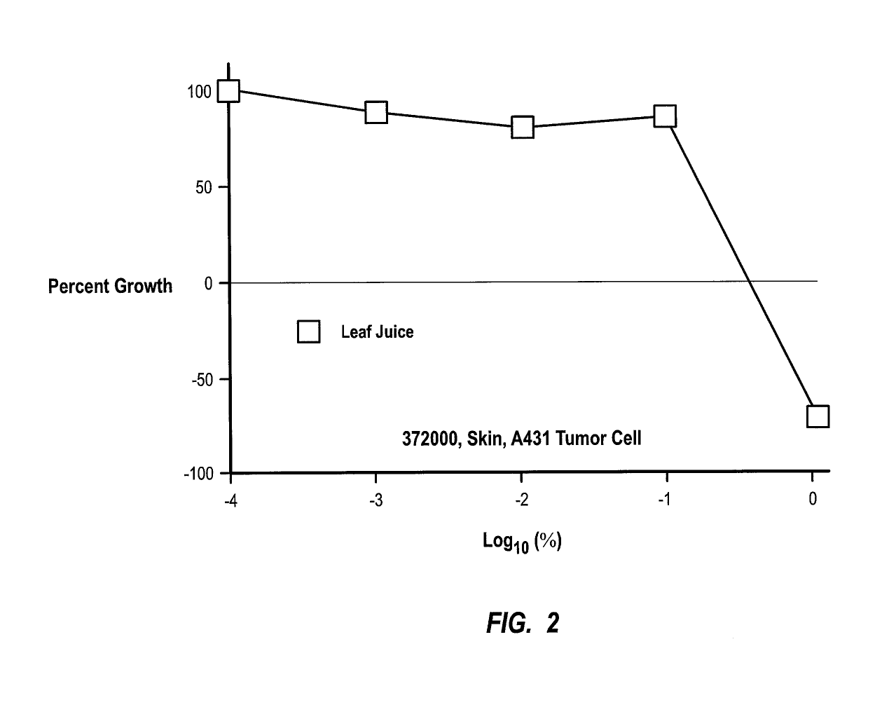

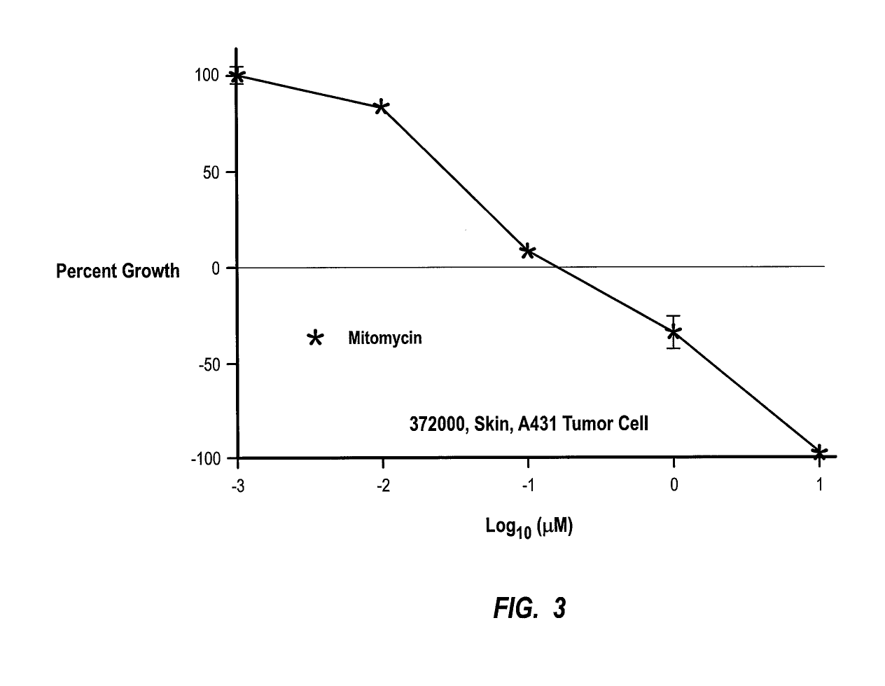

The following tables and FIGS. 1-3 summarize the results of the assay.

### Effect of Morinda Citrifolia Test Substances on the Growth of A431 Skin Tumor Cells¹

TABLE 7

| | | Percent Grown |
|---|---|---|
| | | (Mean = SEM, n = 2) |
| Test | Assay | Concentration (μl/ml) |

| Substance | Name | ^{A}Blank | ^{B}Time₀ | ^{C}Vehicle | 100 | 10 | 1 | 0.1 | 0.01 |
|---|---|---|---|---|---|---|---|---|---|
| Leaf Extract | Skin | −100 | 0 | 100 | 46 +/ | 72 | 89 | 88 + | 101 +/ |
| | | | | | −1 | +/− | +/− | /−3 | −7 |
| | | | | | | 5 | 1 | | |
| Roast Leaf | Skin | −100 | 0 | 100 | 72 +/ | 79 | 87 | 91 + | 99 +/− |
| | | | | | −5 | +/− | +/− | /−6 | 5 |
| | | | | | | 4 | 2 | | |

TABLE 8

Percent Grown

| | | |
|---|---|---|
| Test | Assay | Concentration (%) |

| Substance | Name | Blank | Time₀ | Vehicle | 1 | 0.1 | 0.01 | 0.001 | 0.0001 |
|---|---|---|---|---|---|---|---|---|---|
| Leaf Juice | Skin | −100 | 0 | 100 | 74 +/− | 83 +/− | 77 +/− | 83 +/− | 94 +/− |
| | | | | | 2 | 2 | 2 | 2 | |
| Roast Leaf | Skin | −100 | 0 | 100 | 72 +/− | 79 +/− | 87 +/− | 91 +/− | 99 +/−5 |
| | | | | | 5 | 4 | 2 | 6 | |

TABLE 9

Percent Grown

| | | |
|---|---|---|
| Test | Assay | Concentration (μM) |

| Substance | Name | Blank | Time₀ | Vehicle | 10 | 1 | 0.1 | 0.01 | 0.001 |
|---|---|---|---|---|---|---|---|---|---|
| Mitomycin | Skin | −100 | 0 | 100 | 97 +/− | 35 +/− | 7 +/− | 82 +/− | 101 +/− |
| | | | | | 0 | 8 | 3 | 3 | 5 |

¹ A decrease of 50% or more (≧50%) in fluorescent intensity relative to vehicle-treated control indicates significant growth inhibition, cytostatic or cytotoxic activity.
^{A} Blank: In duplicate, average fluorescent intensity of alamarBlue in medium without cells after 3-day incubation period relative to time₀ (transformed and recorded as −100%).
^{B} Time₀: In duplicate, average fluorescent intensity of alamarBlue in medium just before exposure of cells to test substance (transformed and recorded as 0%).
^{C} Vehicle: In duplicate, average fluorescent intensity of alamarBlue in medium containing cells and added vehicle after 3-day incubation period relative to time₀ (transformed and recorded as 100%).

### IC₅₀, TGI and LC₅₀ Values of Morinda Citrifolia Test Compounds²

TABLE 10

| Compound | Assay Name | ^{a}IC₅₀ | ^{b}TGI | ^{c}LC₅₀ |
|---|---|---|---|---|
| Leaf Extract | Tumor, Skin | 76 μg/ml | >100 μg/ml | >100 μg/ml |
| Leaf Juice | Tumor, Skin | 0.20% | 0.36% | 0.65% |
| Roast Leaf | Tumor, Skin | >100 μg/ml | >100 μg/ml | >100 μg/ml |
| Mitomycin | Tumor, Skin | 0.035 μM | 0.19 μM | 1.0 μM |

²A semi-quantitative determination of IC50, TGI and LC50 was carried out by nonlinear regression analysis using GraphPad Prism (GraphPad Software, USA).
^{a}ID₅₀ (50% Inhibition Concentration): Test compound concentration where the increase from time₀ in the number or mass of treated cells was only 50% as much as the corresponding increase in the vehicle-control at the end of experiment.
^{b}TGI (Total Growth Inhibition): Test compound concentration where the number or mass of treated cells at the end of experiment was equal to that at time₀.
^{c}LC₅₀ (50% Lethal Concentration): Test compound concentration where the number or mass of treated cells at the end of experiment was half that at time₀.

The following figures are concentration-response curves for inhibiton of growth in A431 human tumor cell line treated with Leaf Extract, Leaf Juice and Roast Leaf.

In summary, some embodiments of the present invention provide using Noni leaf juice and Noni leaf extract to inhibit: HMG-CoA Reductase; PDE3 and PDE4; 5-LOX and 15-LOX; XO; GABA and the growth of the second most common human skin cancer cell line, for the purpose of: alleviating pain and inflammation; treating prostate cancers; lowering cholesterol levels; counteracting Diabetes Type II; maintaining the highest possible integrity of cellular interactions in the brain resulting in an undisturbed neural function, (i.e., neuroprotection); ameliorating the effects of asthma and allergies; improving energy; improving insulin secretion; decreasing kidney stone accumulation; alleviating the effects of gout; minimizing convulsions related to epilepsy and other seizure disorders; and providing palliative effects to those addicted to drugs.

### Example 4

In example 4, the effects of ethanolic extracts of Noni leaf juice and fresh squeezed Noni leaf juice on human MMPs were assayed. The following table summarize results of these studies, wherein TNL-3 is ethanolic extracts of the Noni leaf and TNLJ-1 is fresh squeezed Noni leaf juice.

Table 11 depicts the percent inefficient of various concentrations of ethanolic extract of Noni leaf and fresh squeezed Noni leaf juice.

TABLE 11

| | | | |
|---|---|---|---|
| | Concentration | Percent | |
| Samples | IC₅₀ | Inhibition | IC₅₀ |

114910 Peptidase, Matrix Metalloproteinase-9 (MMP-9)

| | | | | | |
|---|---|---|---|---|---|
| TNL₃ | 143733 hum | 2 | 1% | 57 | 0.831% |
| | | 2 | 0.5% | 58 | |
| | | 2 | 0.1% | 25 | |

Thromoxane Synthase Inhibitor

| | | | | | |
|---|---|---|---|---|---|
| TNLJ1 | 143976 hum | 2 | 1% | 57 | 0.831% |
| | | 2 | 0.5% | 31 | |
| | | 2 | 0.1% | 3 | |

114110 Peptidase, Matrix Metalloproteinase-1 (MMP-1)

| | | | | | |
|---|---|---|---|---|---|
| TNL₃ | 143729 hum | 2 | 1% | 76 | 0.517% |
| | | 2 | 0.5% | 41 | |
| | | 2 | 0.1% | 18 | |

114210 Peptidase, Matrix Metalloproteinase-1 (MMP-2)

| | | | | | |
|---|---|---|---|---|---|
| TNL₃ | 143730 hum | 2 | 1% | 85 | 0.234% |
| | | 2 | 0.5% | 61 | |
| | | 2 | 0.1% | 33 | |

114310 Peptidase, Matrix Metalloproteinase-3 (MMP-3)

| | | | | | |
|---|---|---|---|---|---|
| TNL₃ | 143731 hum | 2 | 1% | 86 | 0.184% |
| | | 2 | 0.5% | 67 | |
| | | 2 | 0.1% | 38 | |

As indicated in proceeding table 11, ethanolic extracts of Noni leaf showed significant inhibition of MMP-9, MMP-1, MMP-2 and MMP-3. Example 4 was based on the parameters lined in Tables 12-17.

TABLE 12
Reference Compounds

| | | | |
|---|---|---|---|
| Peptidase, Matrix | TIMP-2 | 5.9 nM | 3.1 nM |
| Metalloproteinase-1 | | | |
| (MMP-1) | | | |
| Peptidase, Matrix | TIMP-2 | 0.95 nM | 1.33 nM |
| Metalloproetinase-2 | | | |
| (MMP-2) | | | |
| Peptidase, Matrix | TIMP-2 | 7.7 nM | 4.02 nM |
| Metalloproteinase-3 | | | |
| (MMP-3) | | | |
| Peptidase, Matrix | TIMP-2 | 2.8 nM | 3.69 nM |
| Metalloproteinase-9 | | | |
| (MMP-9) | | | |
| Thromboxane | 1-(7-Carboxyheptyl)- | 10 nM | 0.0181 μM |
| Synthase | Imidazole | | |

TABLE 13
Peptidase, Matrix Metalloproteinase-1 (MMP-1)

| | |
|---|---|
| Source: | Human rheumatoid synovial fibroblast |
| Substrate: | 4 μM Mca-Pro-Leu-Gly-Leu-Dpa-Ala-Arg- |
| | NH₂ |
| Vehicle: | 1% DMSO |
| Pre-Incubation Tim/Temp: | 60 Minutes @ 37° C. |
| Incubation Time/Temp: | 2 hours @ 37° C. |
| Incubation Buffer: | 50 mM MOPS, pH 7.2, 10 mM CaCl₂, |
| | 2H₂O, 10 μM ZnCl₂, 0.05% Brij 35 |
| Quantitation Method: | Spectrofluorimetric quantitation of Mca- |
| | Pro-Leu-Gly |
| Significance Criteria: | ≧50% of max stimulation or inhibition |

TABLE 14
Peptidase, Matrix Metalloproteinase-3 (MMP-3)

| | |
|---|---|
| Source: | Human recombinant |
| Substrate: | 4 μM Mca-Pro-Leu-Gly-Leu-Dpa-Ala-Arg- |
| | NH₂ |
| Vehicle: | 1% DMSO |
| Pre-Incubation Tim/Temp: | 60 Minutes @ 37° C. |
| Incubation Time/Temp: | 2 hours @ 37° C. |
| Incubation Buffer: | 50 mM MOPS, pH 7.2, 10 mM CaCl₂, |
| | 2H₂O, 10 μM ZnCl₂, 0.05% Brij 35 |
| Quantitation Method: | Spectrofluorimetric quantitation of Mca- |
| | Pro-Leu-Gly |
| Significance Criteria: | ≧50% of max stimulation or inhibition |

TABLE 15
Thromboxane Synthase

| | |
|---|---|
| Source: | Human platelets |
| Substrate: | 10 μM Prostaglandin H₂(PGH₂) |
| Vehicle: | 1% DMSO |
| Pre-Incubation Tim/Temp: | 15 minutes @ 25° C. |
| Incubation Time/Temp: | 3 minutes @ 25° C. |
| Incubation Buffer: | 10 mM Tris-KCl, pH 7.4 |
| Quantitation Method: | EIA quantitation of Thromboxane B₂ |
| | (TxB₂₎ |
| Significance Criteria: | ≧50% of max stimulation or inhibition |

TABLE 16
Peptidase, Matrix Metalloproteinase-2 (MMP-2)

| | |
|---|---|
| Source : | Human recombinant |
| Substrate: | 4 μM Mca-Pro-Leu-Gly-Leu-Dpa-Ala-Arg- |
| | NH₂ |
| Vehicle: | 1% DMSO |
| Pre-Incubation Tim/Temp: | 60 Minutes @ 37° C. |
| Incubation Time/Temp: | 2 hours @ 37° C. |
| Incubation Buffer: | 50 mM MOPS, pH 7.2, 10 mM CaCl₂, |
| | 2H₂O, 10 μM ZnCl₂, 0.05% Brij 35 |
| Quantitation Method: | Spectrofluorimetric quantitation of Mca- |
| | Pro-Leu-Gly |
| Significance Criteria: | ≧50% of max stimulation or inhibition |

TABLE 17
Peptidase, Matrix Metalloproteinase-9 (MMP-9)

| | |
|---|---|
| Source: | Human recombinant |
| Substrate: | 4 μM Mca-Pro-Leu-Gly-Leu-Dpa-Ala-Arg- |
| | NH₂ |
| Vehicle: | 1% DMSO |
| Pre-Incubation Tim/Temp: | 60 Minutes @ 37° C. |
| Incubation Time/Temp: | 2 hours @ 37° C. |
| Incubation Buffer: | 50 mM MOPS, pH 7.2, 10 mM CaCl₂, |
| | 2H₂O, 10 μM ZnCl₂, 0.05% Brij 35 |
| Quantitation Method: | Spectrofluorimetric quantitation of Mca- |
| | Pro-Leu-Gly |
| Significance Criteria: | ≧50% of max stimulation or inhibition |

### Example 5

In Example 5, the effects of ethanolic extracts of Noni leaf were studied. The following table summarize the results of these studies wherein TNL-3 is the ethanolic extract of the Noni leaf. As illustrated in the figures below, ethanolic extract of Noni leaf was shown to be effective in inhibiting bacteria involved in causing acne in humans.

TABLE 18
Results

621000 Propionibacterium acnes (ATCC 6919)

| | | | | | | |
|---|---|---|---|---|---|---|
| TNL₃ | Anaerobes | vit | 2 | 10% | +/−1 | + |
| | | | 2 | 5% | +/−1 | + |
| | | | 2 | 2.5% | +/−1 | − |
| | | | 2 | 1% | +/−1 | − |
| | | | 2 | 0.5% | +/−1 | − |
| | | | 2 | 0.1% | +/−1 | − |
| | | | 2 | 0.05% | +/−1 | − |
| | | | 2 | 0.025 | +/−1 | − |

The foregoing results required utilizing the following materials, methods and reference content.

TABLE 19
Methods and Materials
621000 Propionibacterium acnes (ATCC 6919)

| | |
|---|---|
| Culture Medium: | Reinforced Clostridial Medium |
| Vehicle: | 1% DMSO |
| Incubation Time/Temp: | 2 days @ 37° C. |
| Incubation Volume: | 3 mL |
| Time of Assessment: | 2 days |
| Quantitation Method: | Turbidity measurement and plating count of |
| | subulture |

TABLE 20
Reference Compound Data-Microbial In Vitro Assays

| Assay Name | Class | Reference Compound | Concurrent |
|---|---|---|---|
| Propionibacterium | Anaerobes | Ampicillin | 0.1 μg/mL |
| acnes (ATCC 6919) | | | |

TABLE 21

| | | | |
|---|---|---|---|
| Sample Size | Concentration | % Inhibition | IC₅₀ |

Glutamate, AMPA

| | | | | | |
|---|---|---|---|---|---|
| TNLJ1 | rat | 2 | 10% | 96 | <1% |
| | | 2 | 5% | 92 | |
| | | 2 | 1% | 64 | |

Glutamate Kinate

| | | | | | |
|---|---|---|---|---|---|
| TNLJ1 | rat | 2 | 10% | 103 | <1% |
| | | 2 | 5% | 95 | |
| | | 2 | 1% | 68 | |

Glutamate, NMDA, Agonism

| | | | | | |
|---|---|---|---|---|---|
| TNLJ1 | rat | 2 | 10% | 131 | <1% |
| | | 2 | 5% | 123 | |
| | | 2 | 1% | 85 | |

Glutamate, NMDA, Glycine

| | | | | | |
|---|---|---|---|---|---|
| TNLJ1 | rat | 2 | 10% | 107 | <1% |
| | | 2 | 5% | 100 | |
| | | 2 | 1% | 86 | |

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

What is claimed is:

1. A method of treating disease in, comprising the steps of: administering
a compound containing an extract from Morinda citrifolia leaves; and
inhibiting an enzyme selected from a list consisting of: 5-Lipoxygenase, 15-Lipoxygenase, HMG-CoA Reductase, PDE3, PDE4, XO, and GABA.

2. The method of claim 1, wherein said inhibition of 5-Lipoxygenase and 15-Lipoxygenase results in an effect selected from a list consisting of: treatment of pain, treatment of inflammation, inhibition of 5-Lipoxygenase and 15-Lipoxygenase provides neuroprotection.

3. The method of claim 1, wherein said inhibition results in a physiological effect selected from a list consisting of: treats prostate cancer, lowers cholesterol, treats diseases associated with Type II Diabetes, improves memory, increases insulin, treats for asthma, treats allergies, treats Gout, decreases kidney stone accumulation, improves cardiovascular health, reduce seizures, minimizes convulsant activity, offers therapeutic effects for a mammal suffering from drug addiction and, reducing skin cancer.

4. The method of claim 1 wherein said compound contains Isoflavones.

5. The method of claim 1, wherein said compound contains sterols.

6. A composition comprising a processed Morinda citrifolia component in an amount between 0.01 and 100% by weight selected from a group consisting of: Morinda citrifolia leaf juice, Morinda citrifolia leaf extract, extracts from Morinda citrifolia seeds, Morinda citrifolia seeds, and defatted pulverized Morinda citrifolia seed powder.

7. The composition of claim 6, wherein the composition inhibits the synthesis of leukotrienes from arachidonic acid involving the inhibition of one or more Lipoxygenase enzymes, selected from a list consisting of 5-Lipoxygenase and 15-Lipoxygenase.

8. The composition of claim 6, wherein the composition inhibits the oxygenation of arachidonic acid into its intermediate constituents.

9. The composition of claim 6 further comprising:
processed Morinda citrifolia fruit juice present in an amount by weight between about 0.1-80%;
processed Morinda citrifolia oil present in an amount by weight between about 0.1-20%; and
a carrier medium present in an amount by weight between about 20-90%.

10. A method for inhibiting a Lipoxygenase enzyme comprising the steps of:
adding a processed Morinda citrifolia component to an alcohol-based solution;
isolating and extracting an active ingredient of said processed Morinda citrifolia component from said solution to obtain a fraction;
introducing said extracted active ingredient into said mammal, wherein said extracted active ingredient inhibits oxygenation of arachidonic acid into its intermediate constituents.

11. The method claim of 10, wherein inhibiting the oxygenation of arachidonic acid is affected by inhibiting a lipoxygenase enzyme selected from a list consisting of 5-Lipoxygenase and 15-Lipoxygenase.

12. The method of claim 10, wherein Cyclooxygenase-2 is also selectively inhibited.

13. The method of claim 10, wherein inhibiting the oxygenation of arachidonic acid into its intermediate constituents is accomplished while maintaining gastric mucosal integrity.

14. The method of claim 10, wherein said Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate.

15. The method of claim 10, wherein said alcohol-based solution is selected from the group consisting essentially of methanol, ethanol, and ethyl acetate, and other alcohol-based derivatives.

16. The method of claim 10, wherein said active ingredient is a soluble alcohol supernatant fraction.

17. The method of claim 10, wherein said active ingredient is Quercetin.

18. The method of claim 10, wherein said Quercetin is present in an amount between 0.01 and 10% by weight.

19. The method of claim 10, wherein said active ingredient is Rutin.

20. The method of claim 10, wherein said Rutin is present in an amount between 0.01 and 10% by weight.
